(19) United States
(12) Patent Application Publication
(10) Pub. No.: US 2022/0130509 A1
(43) Pub. Date: Apr. 28, 2022

(54) IMAGE-PROCESSING METHODS AND SYSTEMS

(71) Applicant: SYLORUS ROBOTICS, Lyon (FR)

(72) Inventors: Romain BUTTIN, Saint Priest (FR); Pierre ROUSSOULY, Saint Cyr Au Mont D'or (FR)

(21) Appl. No.: 17/430,917

(22) PCT Filed: Feb. 17, 2020
(86) PCT No.: PCT/EP2020/054055

§ 371 Date: Aug. 13, 2021

(30) Foreign Application Priority Data
Feb. 18, 2019 (FR) 1901615

(51) Int. Cl. G16H 20/40; G06T 15/08; G06T 7/68; G06T 7/73; G16H 30/20

(52) U.S. Cl. CPC G16H 20/40; G06T 15/08; G06T 7/68; G06T 7/73; G06T 2207/10081; G06T 2200/24; G06T 2210/41; G06T 2207/30204; G06T 2207/10121; G16H 30/20

(57) ABSTRACT

This image-processing method comprises steps of: defining, in a three-dimensional digital image of a target object, a plurality of observation directions passing through the three-dimensional digital image and emanating from a predefined observation point; for each observation direction, calculating a resulting value from the respective brightness values of the voxels of the digital image that are passed through by said observation direction; constructing a two-dimensional digital image whose pixel brightness values correspond to the calculated resulting values.

The present invention relates to image-processing methods and systems, particularly for planning a surgical operation.

Three-dimensional X-ray medical imaging techniques, such as computerized tomography (“CT-Scan”), enable measurement of the absorption of X-rays by anatomical structures of a patient and then reconstruction of digital images to visualize said structures.

Such methods can be used during surgical operations, for example to prepare and facilitate the placement of a surgical implant by a surgeon or by a surgical robot.

According to an illustrative and non-limiting example selected from multiple possible applications, these methods may be used during an operation for surgical treatment of a patient's spine, during which one or more spinal implants are placed, for example to perform arthrodesis of a segment of several vertebrae.

Such spinal implants usually include pedicle screws, i.e. screws placed in the pedicles of the patient's vertebrae. The surgical procedures required for the placement of these spinal implants, and particularly for the placement of the pedicle screws, are difficult to perform due to the small size of the bony structures where the implants are to be anchored, and due to the risk of damaging nearby critical anatomical structures such as the spinal cord.

In practice, these surgical procedures are currently performed by orthopedic and neuro-orthopedic surgeons who, after having cleared posterior access to the vertebrae, use ad hoc tools on the latter, in particular bone drilling and screwing tools.

To facilitate these procedures and reduce the risk of damage to the vertebrae or surrounding anatomical structures, and to place the implant in the right place, it is possible to use an intraoperative computer navigation system or a surgical robot.

It is then necessary to first define virtual target marks on the CT images acquired, representing a target position to be taken by each pedicle screw on each vertebra. The target marks are then displayed by the navigation computer system to guide the surgeon, or are used by the surgical robot to define the trajectory of an effector tool carried by a robot arm.

However, it is particularly difficult to manually place a target mark for each vertebra from the CT images acquired. One reason is that it requires manually identifying the most appropriate cutting planes by iteratively reviewing them. The images acquired are usually displayed to an operator as two-dimensional images corresponding to different anatomical cutting planes. The operator must review a large number of images corresponding to different orientations before being able to find a specific orientation that provides a suitable cutting plane from which to define an appropriate target mark.

This requires a great deal of time and experience and is still subject to misjudgment, especially since all of this takes place during surgery, so the time available for this task is limited.

The problem is exacerbated if the patient suffers from a pathology that deforms the spine in several spatial dimensions, such as scoliosis, because the position of the vertebrae can vary considerably from one vertebra to another, which makes the process of identifying the appropriate cutting planes even more time-consuming and complex.

These problems are not exclusive to the placement of spinal implants and can also occur in connection with the placement of other types of orthopedic surgical implants, e.g. for pelvic surgery or, more generally, any surgical implant that needs to be at least partially anchored in a bony structure.

Therefore, there is a need for image processing methods and systems to facilitate the positioning of target marks in intraoperative imaging systems for the placement of surgical implants.

Aspects of the invention aim to remedy these drawbacks by providing a method for automatic planning of a surgical operation according to claim 1.

With the invention, the pixel values of the resulting image are representative of the material density of the target object that has been imaged.

In the case where the imaged object is a bone structure, the resulting image constructed from the acquired images allows for immediate visualization of the bone density of said structure, and in particular visualization of the contrast between areas of high bone density and areas of low bone density within the bone structure itself.

As such, it is easier and faster for an operator to identify a preferred area for insertion of a surgical implant, particularly a surgical implant that must be at least partially anchored in the bone structure.

In particular, in the case where the bone structure is a patient's vertebra, then the bone density information allows an operator to more easily find the optimal cutting plane for each vertebra. Once this cutting plane is identified, the operator can easily define a target mark indicating the direction of insertion of a pedicle screw. In particular, the invention allows the operator to more easily and quickly find where to place the target mark, for example when areas of high bone density are to be preferred.

According to advantageous but not mandatory aspects, such a method may incorporate one or more of the following features, taken alone or in any technically permissible combination:
The three-dimensional digital image is an X-ray image derived from a computed tomography process, with voxel brightness values of the three-dimensional digital image being associated with material density values of the target object.
The method further comprises the steps of:
   acquiring a new position of the observation point;
   in the acquired three-dimensional digital image, defining a plurality of new observation directions through the three-dimensional digital image and emanating from the new observation position; and
   for each observation direction, calculate a new resulting value from the respective brightness values of the voxels of the digital image crossed by the new observation directions;
   constructing a new two-dimensional digital image whose pixel brightness values correspond to the new resulting values calculated.
The method further comprises the calculation of at least one target position of a surgical robot, or even a target trajectory of a surgical robot, from the acquired position of said virtual reference frame.
The calculation of at least one target position or a target trajectory comprises the calculation of the coordinates of the virtual reference frame in a geometrical reference frame linked to a surgical robot from the coordinates of said virtual reference frame in a geometrical reference frame specific to the digital image.
The method also includes steps consisting of:
   after the acquisition of a position of a virtual reference frame, called first virtual reference frame, acquiring coordinates of an axis of symmetry defined on a portion of the two-dimensional digital image by the operator by means of the human-computer interface;
   automatically calculating the position of a second virtual frame of reference by symmetry of the first virtual frame of reference in relation to the defined axis of symmetry.
A calibration marker is placed in the field of view of the imaging device alongside the target object, at least a portion of the marker being made of a material with a predefined material density, so that a part of the three-dimensional digital X-ray image generated includes the image of the calibration marker;

the method further comprising a calibration step in which density values are automatically associated to the brightness values of the pixels of the two-dimensional digital image, automatically determined from the brightness values of a subset of pixels of the same image associated to the portion of the marker made of the material with the predefined material density.

According to another aspect of the invention, a medical imaging system, in particular for a robotic surgery installation, is configured to implement steps of:
acquiring a three-dimensional digital fluoroscopic image of a target object by means of a medical imaging device;
constructing a two-dimensional digital image from the three-dimensional digital fluoroscopic image using an image processing method comprising the steps of: defining the shape of the target object in the three-dimensional digital image; and
   defining a plurality of observation directions in the three-dimensional digital image, through the three-dimensional digital image and emanating from a predefined observation point;
   for each observation direction, calculating a resulting value from the respective brightness values of the voxels of the digital image traversed by said observation direction, the resulting value for each observation direction being calculated as equal to the product of the inverse of the brightness values of the traversed voxels;
   constructing a two-dimensional digital image whose pixel brightness values correspond to the calculated resulting values;
   then acquiring the position of at least one virtual marker defined on the two-dimensional digital image by an operator by means of a human-computer interface.

The invention will be better understood and other advantages thereof will become clearer in light of the following description of an embodiment of an image processing method given only as an example and made with reference to the attached drawings, in which:

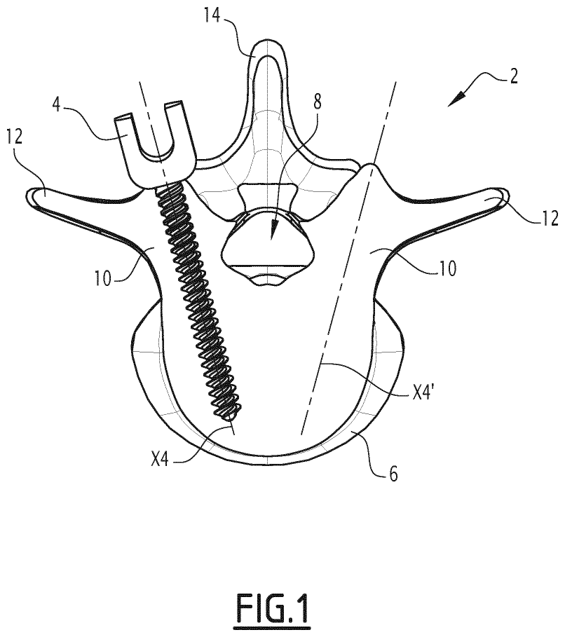

FIG. 1 schematically represents a human vertebra in an axial section plane;

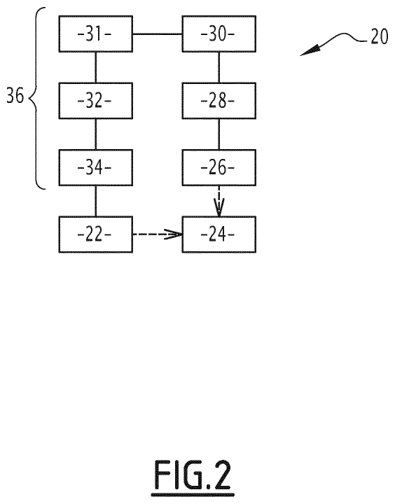

FIG. 2 schematically represents a computer system according to an embodiment of the invention comprising an image processing system and a surgical robot;

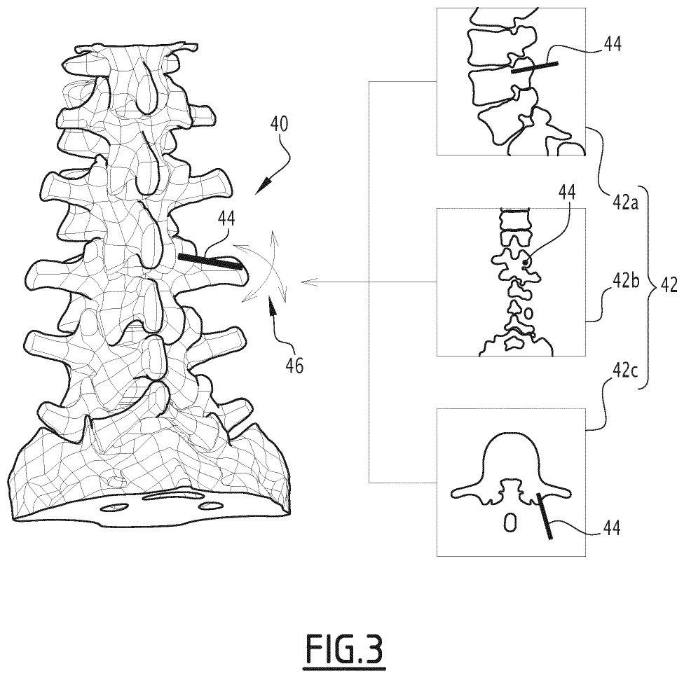

FIG. 3 schematically represents a target marker positioned in a portion of a human spine as well as images of said portion of the spine in anatomical sectional planes on which the target marker is displayed;

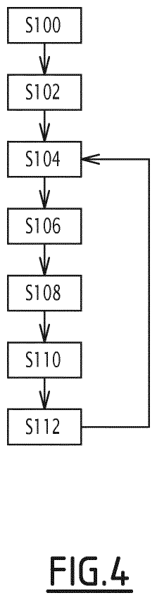

FIG. 4 is a flow diagram of an image processing method according to embodiments of the invention;

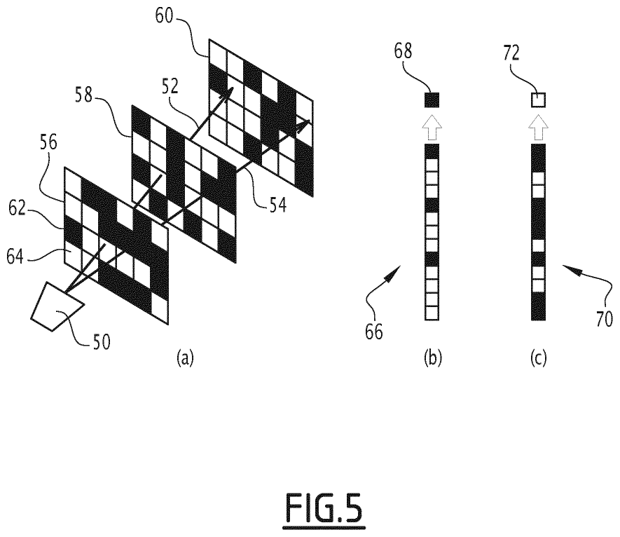

FIG. 5 schematically represents the construction of a resulting image from images acquired by tomography during the process of FIG. 4;

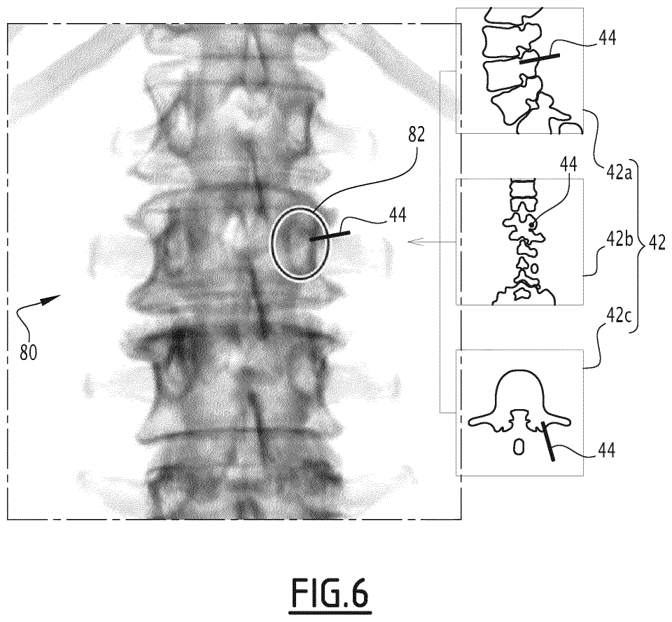

FIG. 6 illustrates an example of an image of a portion of a human spine in a frontal view reconstructed using the method of FIG. 4, as well as images of said portion of the spine in anatomical cross-sectional planes on which the target marker is displayed;

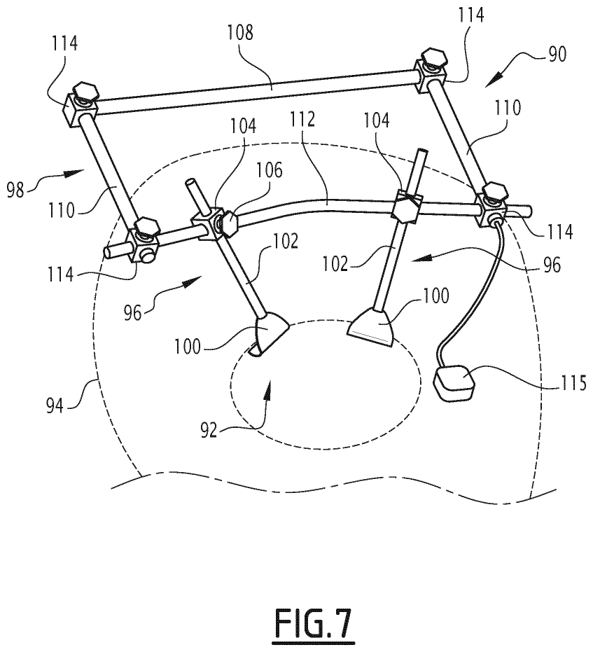

FIG. 7 schematically represents a retractor forming part of the system of FIG. 2;

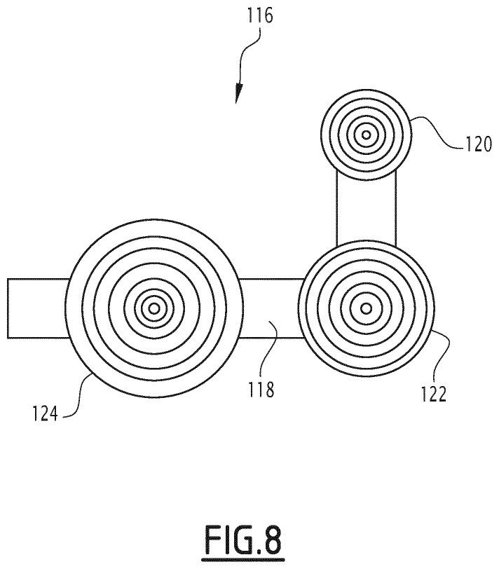

FIG. 8 schematically represents a registration target;

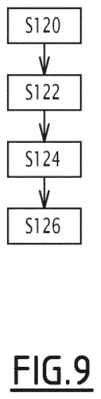

FIG. 9 is a flow diagram of a method of operation of a surgical robot according to embodiments for placing a surgical implant.

The following description is made by way of example with reference to an operation for surgical treatment of a patient's spine in which one or more spinal implants are placed.

The invention is not limited to this example and other applications are possible, including orthopedic applications, such as pelvic surgery or, more generally, the placement of any surgical implant that must be at least partially anchored in a bone structure of a human or animal patient, or the cutting or drilling of such a bone structure. The description below can therefore be generalized and transposed to these other applications.

FIG. 1 shows a bone structure 2 into which a surgical implant 4 is placed along an implantation direction X4.

For example, the bone structure 2 is a human vertebra, shown here in an axial cross-sectional plane.

The implant 4 here includes a pedicle screw inserted into the vertebra 2 and aligned along the implantation direction X4.

This pedicle screw is referred to as “4” in the following.

The vertebra 2 has a body 6 with a canal 8 passing through it, two pedicles 10, two transverse processes 12 and a spinous process 14.

The implantation direction X4 extends along one of the pedicles 10.

The reference X4′ defines a corresponding implantation direction for another pedicle screw 4 (not shown in FIG. 1) and which extends along the other pedicle 10, generally symmetrically to the direction X4.

A notable difficulty arising during implant placement surgery 4 is determining the implantation directions X4 and X4′. The pedicle screws 4 should not be placed too close to the canal 8 or too close to the outer edge of the body 6 so as not to damage the vertebra 2, nor should they be driven too deep so as not to protrude from the anterior body, nor should they be too short so as not to risk being accidentally expelled. One aspect of the process described below is to facilitate this determination prior to implant placement.

FIG. 2 shows a robotic surgical installation 20 having a robotic surgery system 22 for operating on a patient 24.

The surgical installation 20 is located in an operating room, for example.

The robotic surgery system 22 includes a robot arm carrying one or more effector tools, for example a bone drilling tool or a screwing tool. This system is simply referred to as surgical robot 22 in the following.

The robot arm is attached to a support table of the surgical robot 22.

For example, the support table is disposed near an operating table for receiving the patient 24.

The surgical robot 22 includes electronic control circuitry configured to automatically move the effector tool(s) through actuators based on a target position or target trajectory.

The installation 20 includes a medical imaging system configured to acquire a three-dimensional digital fluoroscopic image of a target object, such as a patient's anatomical region 24.

The medical imaging system includes a medical imaging device 26, an image processing unit 28, and a human-computer interface 30.

For example, the apparatus 26 is an X-ray computed tomography apparatus.

The image processing unit 28 is configured to drive the apparatus 26 and to generate the three-dimensional digital fluoroscopic image from radiological measurements made by the apparatus 26.

For example, the processing unit 28 includes an electronic circuit or computer programmed to automatically execute an image processing algorithm, such as by means of a microprocessor and software code stored in a computer-readable data storage medium.

The human-computer interface 30 allows an operator to control and/or supervise the operation of the imaging system.

For example, the interface 30 includes a display screen and data entry means such as a keyboard and/or or touch screen and/or a pointing device such as a mouse or stylus or any equivalent means.

For example, the installation 20 includes an operation planning system comprising a human-computer interface 31, a planning unit 32, and a trajectory calculator 34, this planning system being referred to herein as 36.

The human-computer interface 31 allows an operator to interact with the processing unit 32 and the computer 34, and even to control and/or supervise the operation of the surgical robot 22.

For example, the human-computer interface 31 comprises a display screen and data entry means such as a keyboard and/or or touch screen and/or a pointing device such as a mouse or a stylus or any equivalent means.

The planning unit 32 is programmed to acquire position coordinates of one or more virtual marks defined by an operator by means of the human-computer interface 31 and, if necessary, to convert the coordinates from one geometric reference frame to another, for example from an image reference frame to a robot reference frame 22.

The trajectory calculator 34 is programmed to automatically calculate coordinates of one or more target positions, to form a target trajectory for example, in particular as a function of the virtual mark(s) determined by the planning unit 32.

From these coordinates, the trajectory calculator 34 provides positioning instructions to the robot 22 in order to correctly place the effector tool(s) for performing all or part of the implant placement steps 4.

The planning unit 32 and the trajectory computer 34 comprise an electronic circuit or a computer with a microprocessor and software code stored in a computer-readable data storage medium.

FIG. 3 shows a three-dimensional image 40 of a target object, such as an anatomical structure of the patient 24, preferably a bony structure, such as a portion of the spine of the patient 24.

For example, the three-dimensional image 40 is automatically reconstructed from raw data, in particular from a raw image generated by the imaging device 26, such as a digital image compliant with the DICOM (“digital imaging and communications in medicine”) standard. The reconstruction is implemented by a computer comprising a graphic processing unit, for example, or by one of the units 28 or 32.

The three-dimensional image 40 comprises a plurality of voxels distributed in a three-dimensional volume and which are each associated with a value representing information on the local density of matter of the target object resulting from radiological measurements carried out by the imaging device 26. These values are expressed on the Hounsfield scale, for example.

High density regions of the target object are opaquer to X-rays than low density regions. According to one possible convention, high density regions are assigned a higher brightness value than low density regions.

In practice, the brightness values may be normalized to a predefined pixel value scale, such as an RGB (“Red-Green-Blue”) encoding scale. For example, the normalized brightness is an integer between 0 and 255.

The three-dimensional image 40 is reconstructed from a plurality of two-dimensional images corresponding to slice planes of the device 26, for example. The distances between the voxels and between the cutting planes are known and may be stored in memory.

For example, from the three-dimensional image 40, the imaging unit 28 calculates and displays, on the interface 30, two-dimensional images 42 showing different anatomical sectional planes of the target object, such as a sagittal section 42a, a frontal section 42b, and an axial section 42c.

A virtual mark 44 is illustrated on the image 40 and may be displayed superimposed on the image 40 and on the images 42a, 42b, 42c.

The virtual marker 44 comprises a set of coordinates stored in the memory, for example, and expressed in the geometric reference frame specific to the image 40.

An operator can modify the orientation of the image 40 displayed on the interface 30, for example by rotating or tilting it, using the interface 31.

The operator can also change the position of the virtual marker 44, as illustrated by the arrows 46. Preferably, the images 42a, 42b, and 42c are then recalculated so that the mark 44 remains visible in each of the anatomical planes corresponding to the images 42a, 42b, and 42c. This allows the operator to have a confirmation of the position of the mark 44.

FIG. 4 illustrates an image processing method automatically implemented by the planning system 36.

Beforehand, a raw image of the target object is acquired using the medical imaging system.

For example, the raw image is generated by the processing unit 28, based on a set of radiological measurements performed by the imaging device 26 on the target object.

In a step S100, the digital image 40 is automatically reconstructed from the acquired raw image.

For example, the raw image is transferred from the imaging system to the planning system 36 via the interfaces 30 and 31.

Then, in a step S102, an observation point is defined relative to the digital image 40, for example by choosing a particular orientation of the image 40 using the human-computer interface 31.

The coordinates of the observation point thus defined are stored in the memory and expressed in the geometric reference frame specific to the image 40.

Then, in a step S104, a plurality of observation directions, also called virtual rays, are defined in the three-dimensional image 40 as passing through the three-dimensional image 40 and emanating from the defined observation point.

In FIG. 5, scheme (a) represents an illustrative example in which an observation point 50 is defined from which two virtual rays 52 and 54 emanate, which travel toward the three-dimensional image 40 and successively traverse a plurality of voxels of the three-dimensional image 40.

Only a portion of the three-dimensional image 40 is shown here, in a simplified manner and for illustrative purposes, in the form of two-dimensional slices 56, 58 and 60 aligned along a line passing through the observation point 50 and each containing voxels 62 and 64 here associated with different brightness values.

The virtual rays 52 and 54 are straight lines that diverge from the observation point 50, so they do not necessarily pass through the same voxels as they propagate through the image 40.

The step S104 can be implemented in a way similar to graphical ray tracing methods, with the difference that the projection step used in ray tracing methods is not used here.

In practice, the number of rays 52, 54 and the number of pixels may be different from that shown in this example.

Returning to FIG. 4, in a step S106, a resulting value for each ray is calculated from the respective brightness values of the voxels of the digital image traversed by said ray.

In the example shown in FIG. 5, scheme (b) represents the set 66 of brightness values of the voxels encountered by ray 52 as it travels from observation point 50. The resulting value 68 is calculated from the set 66 of brightness values.

Similarly, scheme (c) represents the set 70 of brightness values of voxels encountered by the ray 52 as it travels from the observation point 50. The resulting value 72 is calculated from the set 70 of brightness values.

Advantageously, the resulting value for each observation direction is calculated as being equal to the product of the inverse of the brightness values of the crossed voxels.

For example, the resulting for each ray is calculated using the following calculation formula:

$${\prod }_{i = 0}^{Max} 1 \times \frac{1}{{ISO}_{i}}$$

In this calculation formula, the subscript “i” identifies the voxels through which the ray passes, “ISOi” refers to the normalized brightness value associated with the i^{th} voxel, and “Max” refers to the maximum length of the ray, imposed by the dimensions of the digital image 40, for example.

With this calculation method, a resulting value calculated in this way will be lower the more the ray has essentially passed through regions of high material density, and will be higher if the ray has essentially passed through regions of low density.

Returning to FIG. 4, in a step S108, a two-dimensional digital image, called the resulting image, is calculated from the calculated resulting values.

The resulting image can then be automatically displayed on the interface screen 31.

In practice, the resulting image is a two-dimensional view of the three-dimensional image as seen from the selected vantage point.

The brightness values of the pixels in the resulting image correspond to the resulting values calculated in the various iterations of step S106.

The brightness values are preferably normalized to allow the resulting image to be displayed in grayscale on a screen.

According to one possible convention (e.g., RGB scale), low resulting regions are visually represented on the image with a darker hue than regions corresponding to high resulting regions.

FIG. 6 shows a resulting image 80 constructed from image 40 showing a portion of the spine of a patient 24.

Preferably, the images 42a, 42b, and 42c are also displayed on the human-computer interface 31 alongside the resulting image 80 and are recalculated based on the orientation given to the image 40.

Through a guided human-computer interaction process, the method thus provides a visual aid to a surgeon or operator to define more easily the target position of a surgical implant using virtual target marks.

In the example of spine surgery, the preferred cutting plane to easily apply the target marks corresponds to an anteroposterior view of the vertebra 2.

The pedicles 10 are then aligned perpendicular to the cutting plane and are easily identified in the resulting image due to their greater density and the fact that their transverse section, which is then aligned in the plane of the image, has a specific shape that is easily identifiable, such as an oval shape, as highlighted by the area 82 in FIG. 6.

As a result, an operator can find a preferred cutting plane more quickly than by observation a sequence of two-dimensional images by changing orientation parameters each time and attempting to select an orientation direction from these cross-sectional views alone.

Optionally, in a step S110, the resulting values are automatically calibrated against a density values scale, so as to associate a density value with each resulting value. In this way, the density can be quantified and not just shown visually in the image 80.

This realignment is accomplished, for example, with the aid of a marker present in the field of view of the apparatus 26 during the X-ray measurements used to construct the image 40, as will be understood from the description made below with reference to FIG. 8.

For example, the marker is placed at the sides of the target object and at least a portion of the marker is made of a material with a predefined material density, so that a portion of the generated three-dimensional digital X-ray image includes the calibration marker image. During calibration, the brightness values of the pixels in the image 80 are automatically associated with density values automatically determined from the brightness values of a subset of pixels in that same image associated with the portion of the marker made of the material with the predefined material density.

Optionally, the observation angle of the resulting image can be changed and a new resulting image is then automatically calculated based on the newly selected orientation. To this end, in a step S112, a new position of the observation point is acquired, for example by means of the interface 31 in response to an operator selection. The steps S104, S106, S108 are then repeated with the new observation point position, to define new observation directions from which new resulting values are calculated to build a new resulting image, which differs from the previous resulting image only by the position from which the target object is seen.

Optionally, on the human machine interface 31, the resulting image 80 may be displayed in a specific area of the screen alternating with a two-dimensional image 42 showing the same region. An operator can alternate between the resulting image view and the two-dimensional image 42, for example if he or she wishes to confirm an anatomical interpretation of the image.

FIG. 9 shows a method for automatically planning a surgical operation, in particular a surgical implant operation, implemented using the system 20.

In a step S120, a three-dimensional digital fluoroscopic image of a target object is acquired by means of the medical imaging system and then a resulting image 80 is automatically constructed and then displayed from the three-dimensional image 40 by means of an image processing method in accordance with one of the previously described embodiments.

Once a resulting image 80 taken in an appropriate cutting plane is displayed, the operator defines the location of the virtual marker using the input means of the interface 31. For example, the operator places or draws a line segment defining a direction and positions of the virtual marker. In a variant, the operator may only point to a particular point, such as the center of the displayed cross section of the pedicle 10. The virtual mark may be displayed on image 80 and/or image 40 and/or images 42. Multiple virtual marks may thus be defined on a single image.

During a step S122, the position of at least one virtual mark 44 defined on the image 80 is acquired, for example by the planning unit 32, by an operator by means of a human-computer interface.

Optionally, during a step S124, after the acquisition of a position of a virtual reference frame, called first virtual reference frame, coordinates of an axis of symmetry defined on a portion of the image 80 by the operator by means of the interface 31 are acquired.

For example, the axis of symmetry is drawn on the image 80 by the operator using the interface 31. Then, the position of a second virtual mark is automatically calculated by symmetry of the first virtual mark in relation to the defined axis of symmetry.

In the case of a vertebra 2, once the X4 direction has been defined, the X4′ direction can thus be determined automatically if the operator believes that the vertebra 2 is sufficiently symmetrical.

One or more other virtual marks may be similarly defined in the remainder of the image once a virtual mark has been defined, between several successive vertebrae of a spine portion for example.

In a step S126, at least one target position, or even a target trajectory of the surgical robot 22 is automatically calculated by the unit 34 from the acquired position of the previously acquired virtual mark. This calculation can take into account the control laws of the robot 22 or a pre-established surgical program.

For example, this calculation comprises the calculation by the unit 34 of the coordinates of the virtual reference frame in a geometric reference frame linked to the surgical robot 22 from the coordinates of said virtual reference frame in a geometric reference frame specific to the digital image.

According to one possibility, the reference frame of the robot 22 is mechanically linked without a degree of freedom to the geometric reference frame of the digital image 40, immobilizing the patient 24 with the support table of the robot 22 for example, which allows a correspondence to be established between a geometric reference frame of the surgical robot and a geometric reference frame of the patient. Here, this immobilization is achieved through spacers connected to the robot support table 22, as explained below.

Optionally, when the calibration step S110 is implemented, the density values can be used when calculating the trajectory or programming parameters of the robot 22. For example, a bone drilling tool will need to apply a higher drilling torque in bone regions for which a higher bone density has been measured.

Once calculated, the positional and/or trajectory coordinates can then be transmitted to the robot 22 to position a tool to perform a surgical operation, including the placement of a surgical implant, or at least to assist a surgeon in performing the surgical operation.

FIG. 7 shows an example of a surgical instrument 90 for immobilizing the patient 24 with the robot support table 22 and including a retractor for pushing back sides of an incision 92 made in the body 94 of the patient 24 including retractor arms 96 mounted on a frame 98.

Each retractor arm 96 comprises a retractor tool 100 mounted at one end of a bar 102 secured to the framework 100 by a fastener 104 adjustable by an adjustment knob 106.

The frame 98 comprises a fastening system by means of which it can be fixedly attached without degrees of freedom to the robot 22, preferably to the support table of the robot 22.

The frame 98 is formed by assembling a plurality of bars, here of tubular shape, these bars comprising in particular a main bar 108 fixedly attached without a degree of freedom to the support table of the robot 22, side bars 110 and a front bar 112 on which the spacer arms 96 are mounted. The bars are fixed together at their respective ends by fixing devices 114 similar to the devices 104.

The frame 98 is arranged to overhang the patient's body 94, and here has a substantially rectangular shape.

Preferably, the frame 98 and the spacer arms 96 are made of a radiolucent material, so as not to be visible in the image 40.

The spacer 96 may be configured to immobilize the patient's spine 24 made accessible by the incision 92, which facilitates linking the patient to the reference frame of the robot 22 and avoiding any movement that might induce a spatial shift between the image and the actual position of the patient.

Optionally, as illustrated in FIG. 8, a calibration marker 116 made of a radiopaque material, i.e., a material that is opaque to X-rays, may be used in the installation 20.

The marker 116 may be attached to the instrument 90, held integral to the frame 98, for example, although this is not required. The marker 116 may be attached to the end of the robot arm, for example.

At least a portion of the marker 116 has a regular geometric shape, so as to be easily identifiable in the images 40 and 80.

For example, the marker 116 includes a body 118, cylindrical in shape for example, and one or more disk- or sphere-shaped portions 120, 122, 124, preferably having different diameters. For example, these diameters are larger than the dimensions of the body 118.

A spherical shape has the advantage of having the same appearance regardless of the observation angle.

At least a portion of the marker 116, preferably those having a recognizable shape, in particular spherical, is made of a material with a predefined material density. In the calibration step S110, the density scale calibration is performed by identifying this marker portion on the image 40 or 80, by automatic pattern recognition or by manual pointing of the shape on the image by the operator through the interface 30.

In a variant, many other embodiments are possible.

The medical imaging system comprising the apparatus 26 and the unit 28 can be used independently of the surgical robot 22 and the planning system 36. Thus, the image processing method described above can be used independently of the surgical planning methods described above. For example, this image processing method can be used for non-destructive testing of mechanical parts using industrial imaging techniques.

The instrument 90 and the image processing method may be used independently of each other.

The instrument 90 may include a movement sensor such as an inertial motion sensor, labeled 115 in FIG. 7, to measure movements of the patient 24 during the operation and correct the calculated positions or trajectories accordingly.

For example, the sensor 115 is connected to the unit 32 via a data link. The unit 32 is programmed to record patient movements measured by the sensor 115 and to automatically correct positions or trajectories of a robot arm based on the measured movements.

The embodiments and variants contemplated above may be combined with each other to generate new embodiments.

1. A method for automatically planning a surgical operation, wherein said method comprises:
constructing a three-dimensional digital fluoroscopic image of a target object by means of a medical imaging device;
constructing a two-dimensional digital image from the three-dimensional digital fluoroscopic image by means of an image processing method; and
acquiring the position of at least one virtual mark defined on the two-dimensional digital image by an operator by means of a human-computer interface
said image processing method comprising:
defining, in a three-dimensional digital image of a target object, a plurality of observation directions passing through the three-dimensional digital image and emanating from a predefined observation point;
calculating a resulting value for each observation direction from the respective brightness values of the digital image voxels passed through by said observation direction
constructing a two-dimensional digital image whose pixel brightness values correspond to the calculated resulting values,
and wherein the resulting value for each observation direction is calculated as equal to the product of the inverse of the brightness values of the voxels passed through.

2. The method according to claim 1, wherein the three-dimensional digital image is an X-ray image from a computed tomography method, the brightness values of voxels of the three-dimensional digital image being associated with material density values of the target object.

3. The method according to claim 1, wherein the image processing method further comprises the steps of:
acquiring a new position of the observation point;
defining, in the acquired three-dimensional digital image, a plurality of new observation directions passing through the three-dimensional digital image and emanating from the new viewpoint position;
calculating a new resulting value for each observation direction from the respective brightness values of the voxels of the digital image voxels passed through by the new observation directions;
constructing a new two-dimensional digital image whose pixel brightness values correspond to the new calculated resulting values.

4. The method according to claim 1, wherein said method further comprises calculating at least one target position of a surgical robot, or even a target trajectory of a surgical robot, from the acquired position of said virtual mark.

5. The method according to claim 1, wherein the calculation of at least one target position or a target trajectory comprises calculating the coordinates of the virtual reference frame in a geometric reference frame linked to a surgical robot from the coordinates of said virtual reference frame in a geometric reference frame specific to the digital image.

6. The method according to claim 1, wherein said method further comprises:
after acquiring a virtual reference frame position, known as the first virtual reference frame, acquiring the coordinates of an axis of symmetry defined on a portion of the two-dimensional digital image by the operator by means of the human-computer interface;
automatically calculating the position of a second virtual frame of reference by symmetry of the first virtual frame of reference in relation to the defined axis of symmetry.

7. The method according to claim 1, wherein a calibration marker is placed in the field of view of the imaging apparatus alongside the target object, at least a portion of the marker being made of a material with a predefined material density, such that a portion of the generated three-dimensional digital fluoroscopic image includes the image of the calibration marker;
and wherein the method further comprises a calibration step wherein are automatically associated with the brightness values of the pixels of the two-dimensional digital image, density values automatically determined from the brightness values of a subset of pixels of this same image associated with the portion of the marker made of the material having the predefined material density.

8. A medical imaging system, wherein said medical imaging system is configured to implement steps of:
acquiring a three-dimensional digital fluoroscopic image of a target object by means of a medical imaging apparatus;
constructing a two-dimensional digital image from the three-dimensional digital fluoroscopic image by means of an image processing method comprising:
defining in the three-dimensional digital image a plurality of observation directions through the three-dimensional digital image and emanating from a predefined observation point;
calculating a resulting value for each observation direction from the respective brightness values of the voxels of the digital image passed through by said observation direction, the resulting value being calculated, for each observation direction, as equal to the product of the inverse of the brightness values of the passed through voxels;
constructing a two-dimensional digital image whose pixel brightness values correspond to the calculated resulting values;
then acquiring the position of at least one virtual marker defined on the two-dimensional digital image by an operator using a human-computer interface.
